(19) United States
(12) Patent Application Publication
(10) Pub. No.: US 2014/0018938 A1
(43) Pub. Date: Jan. 16, 2014

(54) PROSTHESIS SYSTEM

(71) Applicants: Thomas Bertels, Duderstadt (DE); Bernard Garus, Einbeck (DE); Thomas Kettwig, Gottingen (DE)

(72) Inventors: Thomas Bertels, Duderstadt (DE); Bernard Garus, Einbeck (DE); Thomas Kettwig, Gottingen (DE)

(73) Assignee: OTTO BOCK HEALTHCARE GmbH, Duderstadt (DE)

(21) Appl. No.: 14/007,557

(22) PCT Filed: Mar. 23, 2012
(86) PCT No.: PCT/EP2012/001280

§ 371 Date: Sep. 25, 2013

(30) Foreign Application Priority Data
Mar. 25, 2011 (DE) 10 2011 015 502.3

(51) Int. Cl. A61F 2/78

(52) U.S. Cl. CPC A61F 2/7812
USPC 623/25

(57) ABSTRACT

The invention relates to a prosthesis system with a liner (2) made of an electrically non-conducting liner material, which is intended to be pulled over an amputation stump, at least one electrode (10), which has at least one contact surface (14), and a prosthesis socket, which is intended to be arranged on the amputation stump, wherein the at least one electrode (10) is arranged on the inner face of the prosthesis socket, at least one area (6) of the liner (2) has a multiplicity of lead-throughs (8) from the inner face (22) to the outer face (24) made of an electrically conducting material arranged in the liner material, and the at least one contact surface (14) of the at least one electrode (10) is intended to come into contact with at least one lead-through (8) during use.

The invention relates to a prosthesis system with a liner made of an electrically non-conducting liner material, which is provided to be pulled over an amputation stump and which has an inner face provided to rest against the amputation stump and an outer face facing away from the inner face, at least one electrode which has at least one contact face, and a prosthesis socket, which is provided to be arranged on the amputation stump after the liner was pulled thereover such that a socket inner face faces the outer face of the liner. The invention moreover relates to a liner and a prosthesis socket for such a prosthesis system.

In the case of a conventional prosthesis system of the type discussed here, the prosthesis socket forms a part which replaces the amputated part of an extremity of a patient. The object of the liner is, inter alia, to form a cushioning intermediate layer between the stump and the inner wall of the prosthesis socket, which intermediate layer adapts to the amputation stump or is adapted thereto.

The prior art has disclosed the practice of recording myoelectric signals by means of at least one electrode. To this end, the electrode is arranged on the skin of the amputation stump. As a result of muscle contractions in the amputation stump, myoelectric electrodes can pick up electric muscle-contraction signals, by means of which the control of corresponding prosthesis functions becomes possible. By way of example, in the case of a forearm and hand prosthesis, this renders it possible to control functions of the hand via these electric signals generated from the muscle contractions in the amputation stump. The prior art has disclosed the myoelectric control for arm and hand prostheses in particular; however, it can also be used for leg and foot prostheses.

Moreover, it may be expedient to determine a surface resistance of the skin of the amputation stump electrically by virtue of, for example, a current flow being measured between two or more electrodes or electrode sections. By way of example, this renders it possible to determine whether the skin within the liner transpires, as a result of which the seat of the liner on the amputation stump, and hence the seat of the prosthesis system, can deteriorate.

Conversely, it may be expedient to transmit electric signals to the skin of the amputation stump, in order, for example, to excite muscle contractions in the amputation stump.

Conventionally, the at least one electrode is directly in the liner of the prosthesis system, since here is the only point at which the prosthesis system, and hence also the electrode, is able to come into contact with the skin of the amputation stump. If myoelectric signals are intended to be picked up, exact positioning of the electrode relative to the muscle which generates the myoelectric signals is very important. Even only a slight displacement of the electrode on the amputation stump already results in the fact that although it still being possible to pick up myoelectric signals, these may differ from the myoelectric signals which can be picked up by the electrode at the correct location on the amputation stump. A control in the prosthesis system, which processes the picked up electric signals and uses these for controlling the prosthesis functions, may not identify the signals picked up by a displaced electrode, and so there may be malfunctions and breakdown of functions of the prosthesis system.

A liner conventionally consists of a non-conducting material, for example an elastomer, a textile-coated elastomer or a 3D-textile. The liner is often placed against the amputation stump in a manner similar to putting on tights. The liner, on which the electrode is conventionally arranged, is generally rolled up for application and placed against the tip of the amputation stump in the rolled-up state. The liner is subsequently unrolled and thus, like tights, pulled over the amputation stump. In the process, the liner can easily be twisted or displaced by a few degrees. As a result of the liner, which is for example made of silicone, resting tightly against the amputation stump, a subsequent correction of the position of the liner relative to the amputation stump can only be carried out with difficulty and with discomfort for the patient and with great effort.

If the electrode is situated on the liner, as known from the prior art, a slight displacement of the liner also results in a slight displacement of the electrode, as a result of which the myoelectric signals picked up by the electrode are no longer the signals expected by the control of the prosthesis system, and so the malfunctions or breakdown of functions of the prosthesis system, as discussed above, may occur.

In order to ensure more exact positioning of the electrode relative to the amputation stump, it is known to provide a cutout, a so-called window, in the liner, the dimensions of which cutout are greater than the electrode to be positioned. The electrode is either attached separately or arranged on the prosthesis socket, which can be positioned much more easily relative to the amputation stump. As a result of the window having larger dimensions than the electrode to be positioned, even a slight displacement of the liner and hence of the window is harmless. The electrode is still positioned in the window of the liner. However, a disadvantage is that an interspace which is not covered by the liner or by the electrode is created between the electrode and the liner. The skin can be compressed in this interspace, and so so-called window edemas may occur.

The prior art moreover discloses that a cost intensive, individual liner is fabricated for every patient, which liner is adapted precisely to the shape of the amputation stump. As a result, exact positioning of the liner, and hence of the electrode arranged thereon, is also possible relative to the amputation stump. However, it is disadvantageous that the production is not only cost intensive but also requires much time, and so the patient may have to wait for weeks for his prosthesis system.

U.S. Pat. No. 5,443,525 has disclosed a liner which is provided for holding myoelectric electrodes. To this end, a flexible soft cushion is adhesively bonded into a window of the liner, into which cushion electrodes have been worked. The electrode arrangement is therefore adhesively bonded to the inside of the liner by the cushion and accessible through the window of the liner such that the myoelectric signals picked up by the electrodes can be transmitted. A disadvantage of this exemplary embodiment is that exact positioning of the electrode relative to the amputation stump is also only possible by exactly positioning the liner. Thus, this also requires a complicated application method of the liner which is uncomfortable to the patient or a cost intensive and time-consuming production of an individual, adapted liner. The arrangement is moreover complicated in production and only has restricted comfort of wear. The window of the liner moreover requires particular sealing complexity if, as is often the case, the liner must have an airtight design in order to keep the liner on the amputation stump with the aid of negative pressure formed in the interior of the liner.

US 2009/0216339 A1 has disclosed a system with which myoelectric signals can be transmitted through a liner. Here, inserts are adhesively bonded into the liner, which inserts have a first part which comes into contact with the skin of the patient, a second part which leads through the liner and a third part which is arranged on the outside of the liner. Here, this third part has a particularly large surface in order to ensure that an electrode with a contact face is brought into contact with this face in a particularly simple and reliable manner. However, it is also disadvantageous in this case that, although the electrode need not be positioned particularly precisely relative to the liner since the inserts have a large area in the third part, the liner must however be positioned exactly relative to the amputation stump so that the myoelectric signals can be picked up at the correct position. This case also requires the production of an individualized, adapted liner.

DE 10 2007 035 409 has disclosed a liner made of a 3D-textile, which has electrodes on the lower side. These can also be worked into the 3D-textile. However, how the electric through-contacting takes place is not disclosed.

The invention is therefore based on the object of developing a generic prosthesis system in such a way that it is possible to pick up electric signals, in particular myoelectric signals, at the right position in a simple and cost-effective manner, which is comfortable to the patient.

The invention achieves the stated object by a generic prosthesis system, which is distinguished by virtue of the at least one electrode being arranged on the outer face of the liner or on the socket inner face of the prosthesis socket, a plurality of feedthroughs which run from the inner face to the outer face and are made of an electrically conducting material in the liner material being arranged in at least one region of the liner and the at least one contact face of the at least one electrode being provided to come into contact with at least one feedthrough when the prosthesis socket is arranged on the amputation stump after the liner was pulled over the latter.

As a result of the at least one electrode being arranged on the outer face of the liner or on the socket inner face of the prosthesis socket, exact positioning of the electrode relative to the amputation stump is possible in a simple and convenient manner.

The liner is equipped with a plurality of feedthroughs in at least one region, which feedthroughs are electrically insulated from one another. Each of these feedthroughs has a face on the inside of the liner and a face on the outside of the liner. These feedthroughs render it possible to pick up electric signals from the amputation stump at the respective position of the feedthrough and route said signal through the liner. When the liner is initially pulled over an amputation stump and when the prosthesis socket is arranged over said liner, the electrode which is arranged on the inside of the prosthesis socket comes into contact with at least one of these feedthroughs. In this manner the electrode is able to pick up the electric signals from the skin of the amputation stump through the liner. The location of this pick up is in this case merely determined by which of the feedthroughs comes into contact with the contact face of the at least one electrode. Hence the position at which the electric signals are picked up is substantially determined by the position of the electrode. A slight shift or twist of the liner relative to the amputation stump is therefore harmless to the position of the signal pick up.

The at least one region in which the liner is provided with a plurality of feedthroughs is advantageously greater than the at least one contact face of the at least one electrode, preferably at least twice the size thereof. This ensures that, even in the case of a relatively large twist or displacement of the liner relative to the amputation stump, and hence also relative to the prosthesis socket, the at least one contact face of the at least one electrode still comes into contact with at least one of the feedthroughs.

It was found to be advantageous if a plurality of electrodes arranged on the socket inner face of the prosthesis socket are provided and if the liner has a plurality of regions with a plurality of feedthroughs. This renders the pickup of electric signals, in particular myoelectric signals, possible at a plurality of points on the amputation stump, as a result of which a larger number of different signals, and hence of different control commands, can be picked up for the prosthesis functions. As a result, prostheses which can have significantly more and more complex functions are possible, and so these are more similar to the body part to be replaced.

Here, each position of the electrode on the socket inside of the prosthesis socket is advantageously associated with a region of the liner in which the latter is equipped with a plurality of feedthroughs. As an alternative to this, the liner can, of course, also be equipped with a plurality of feedthroughs over its entire area. However, in most cases this is disadvantageous for economic reasons.

The plurality of feedthroughs preferably have an integral design with the liner. By way of example, this can be achieved by virtue of a conducting section being inserted into the liner material, which is preferably a polymer, e.g. silicone, e.g. before polymerization. During polymerization of the material of the liner, the liner is connected with the conducting section to form a uniform part such that an integral liner is formed. As an alternative to this, the plurality of feedthroughs can also be formed by rivets or screws introduced into the liner material. By way of example, these rivets or screws can consist of copper, titanium or a conducting plastic. Alternatively, it is furthermore possible to inject a conducting plastic through the liner. By way of example, nozzles are routed through the material for this purpose in the case of a liner made of a 3D-textile. In the case of a liner made of a polymer material, the material is pierced or holed and the nozzles are routed through the holes generated thus. The nozzles themselves can also be designed to pierce the material. The conducting plastic is then introduced by the nozzles.

It is advantageous in all cases if every one of the plurality of feedthroughs is an electric conductor which is perpendicular to the inner face of the liner and hence also perpendicular to the skin of the patient at the amputation stump. As a result of this, the position of picking up the electric signals corresponds to the position of the respective feedthrough relative to the amputation stump and hence also to the position of the electrode. Since the electrode can be positioned very easily relative to the amputation stump, a displacement, twist or shift of the liner relative to the amputation stump is particularly harmless in this embodiment.

Advantageously, the at least one electrode is displaceably arranged on the outside of the liner or on the socket inner face. As a result of this, the position of the electrode relative to the amputation stump can be readjusted, even after adapting the prosthesis socket to the amputation stump, in order to achieve the optimum position in this case.

The at least one electrode preferably has a plurality of contact faces. This renders it possible to pick up a larger number of electric signals and thereby achieve more complex functions and control signals for the prosthesis system.

In a preferred embodiment, a face of each feedthrough on the outside is greater than or equal to one square millimeter for the contact with the at least one contact face of the at least one electrode. This size is sufficient to ensure secure contacting. In particular, permanent contacting can be ensured by the pressure of the electrode on the liner. At the same time, the face is small enough to space two neighboring electrodes so far from one another that they are electrically insulated from one another. As a result, a short circuit is avoided and the functionality is maintained.

A liner according to the invention for a prosthesis system as described above is distinguished, in particular, by virtue of a face of each feedthrough on the inner face of the liner having exactly the same size as the face of each feedthrough on the outer face of the liner.

A prosthesis socket according to the invention can be used in a prosthesis system as described above and is distinguished, in particular, by virtue of the at least one electrode being arranged on the socket inside. Here, a recess for the electrode to be arranged can by all means also be present on the socket inside. Within the meaning of this invention, the phrasing that the electrode is attached or arranged on the socket inside means that, in particular, the electrode can come into contact with the feedthroughs which are provided in the liner.

Using a prosthesis system according to the invention, individual fabrication of the liner for a myoelectric supply of a prosthesis system may no longer be required such that said liner can be pre-manufactured in standard sizes and/or such that the liner is configured in such a way that the liner takes the shape of the stump by the elasticity thereof and therefore also covers intermediate sizes. Moreover, this standard liner need not necessarily be put on with a precise fit and exact alignment. A twist or a displacement of the liner by a few degrees is possible by all means and harmless to the quality of the picked up electric signals. As a result of arranging the plurality of feedthroughs, there always is electric contact with the contact face of the at least one electrode. The regions in which the liner is provided with the plurality of feedthroughs are preferably so large that the liner can be shortened individually, particularly in terms of its length, without a complete one of these regions being removed. Hence the liner can be individually adapted, at least to a small extent, by simple means, without the quality of the picked up myoelectric signals being impaired. Moreover, apart from the cutting to length, the liner is not additionally damaged.

A further advantage consists of the fact that an orthopedic technician, who wishes to use a liner as described above as a standard liner, is not restricted to one electrode position on the socket inside of the prosthesis socket. Here, the position of the electrodes is freely selectable within certain boundaries, and so the position that is optimum for the patient can be selected.

The prosthesis system has a breathable design in a preferred embodiment. The liner then is a 3D knitted spacer fabric, through which e.g. rivets or a conducting plastic are routed as electric contacts. This can occur particularly easily as a result of the textile structure of the liner. The prosthesis socket, in particular an inner socket, encompasses the volume and ensures the desired orientation of the electrode on the body. Here, the inner socket preferably has openings for ensuring breathing, i.e. the air circulation through these openings.

In a preferred embodiment of a prosthesis system, the non-conducting liner material is a hydrophobic material. As an alternative or in addition thereto, the electrically conducting material of the feedthroughs advantageously is a hydrophilic material.

In order to achieve good electric contact between the feedthroughs on the inside of the liner and the skin of the wearer of the prosthesis system, it is advantageous if the contact point is wetted. This can be achieved particularly easily with feedthroughs made of a hydrophilic electrically conducting material. If a hydrophobic material is utilized as liner material at the same time, this can achieve a liner which can be applied particularly easily and reproducibly, and hence a prosthesis system can be achieved. Thus, for example, it is possible to wet the liner prior to application, e.g. fill it with water. The water is subsequently removed from the liner, for example poured out. The hydrophobic, i.e. water repellent, material of the liner is almost completely dry thereafter while the hydrophilic, i.e. water attracting, material of the feedthroughs remains damp. As a result of this, precisely that part on the inside of the liner which is required for good electric contacting is damp while the remaining remainder is dry such that there is comfortable, clean comfort of wear. Moreover, such a liner can be cleaned particularly easily and the skin tolerance is increased.

In particular, what the hydrophobic and hydrophilic property of the corresponding areas of the liner inside achieves is that at least almost no residual liquid is present on the hydrophobic components such that the risk of short circuits and unwanted transmissions of electric signals is reduced or even completely removed. This enables a cleaner signal transmission.

It is naturally possible to use active and passive electrodes, i.e. with an integrated or separate amplifier element.

As an alternative to an inherently hydrophilic or hydrophobic material, provision can naturally also be made for a hydrophilic or hydrophobic coating provided in the corresponding region. This means that the inside of the liner at which no feedthroughs are provided can be coated with a hydrophobic material. Alternatively, or in addition thereto, the inside of the liner can be coated with a hydrophilic material in the region in which the feedthroughs are provided; optionally, it is possible for only the inside of the feedthroughs to be coated with said hydrophilic material.

By way of example, the hydrophobicity of materials can be specified by the contact angle. The hydrophobicity of the respective surface increases with the contact angle. Here, surfaces with a contact angle of less than 90° are referred to as hydrophilic and materials with a contact angle of more than 90° are referred to as hydrophobic.

An exemplary embodiment of the present invention is explained in more detail below with the aid of a drawing. In detail:

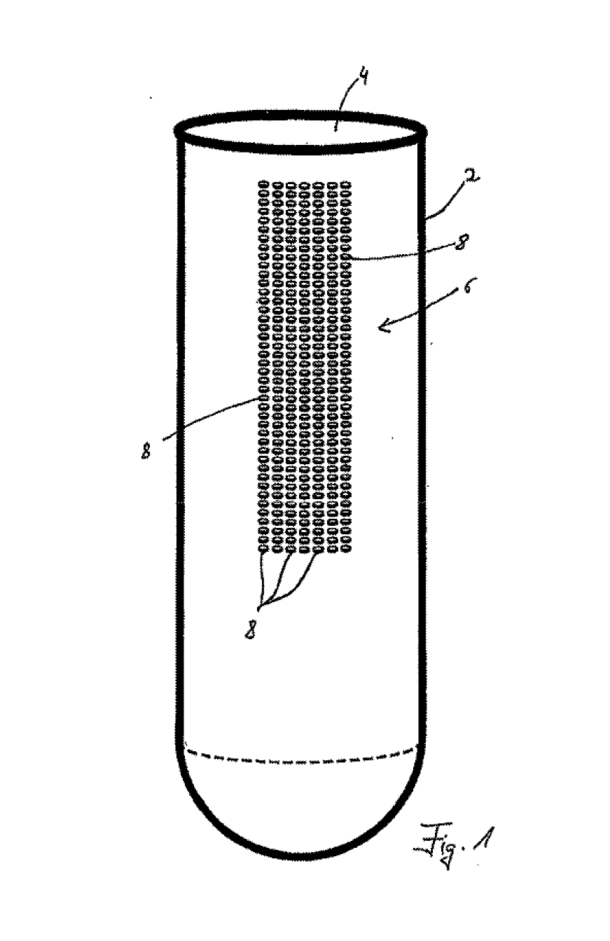

FIG. 1 shows a liner with a multiplicity of feedthroughs for a prosthesis system in accordance with one exemplary embodiment of the present invention,

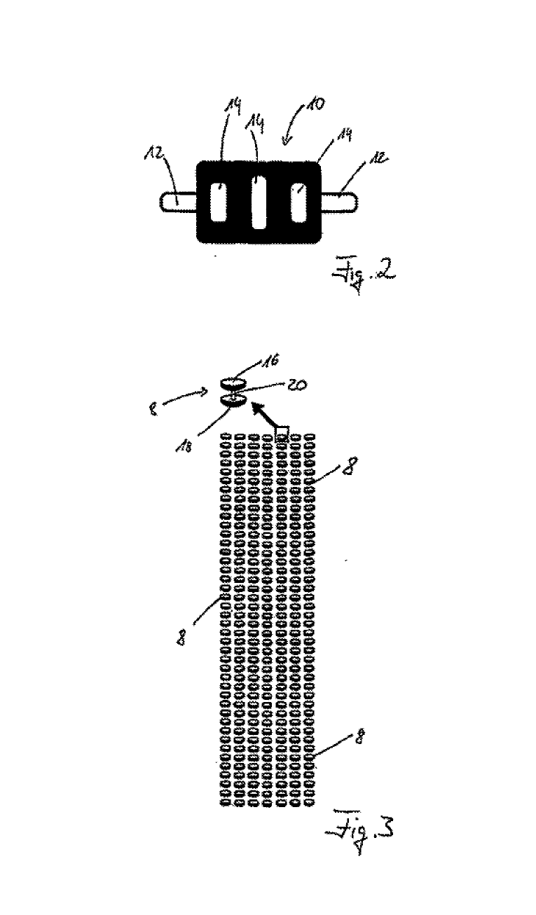

FIG. 2 shows the schematic depiction of an electrode for a prosthesis system in accordance with one exemplary embodiment of the present invention,

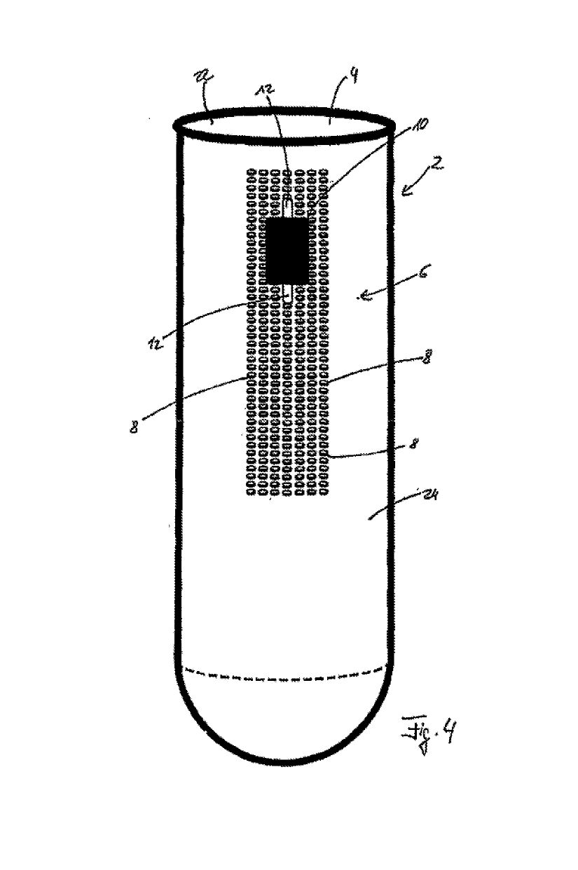

FIG. 3 shows the arrangement of a plurality of feedthroughs with a magnified depiction of a feedthrough for a prosthesis system in accordance with a further exemplary embodiment of the present invention,

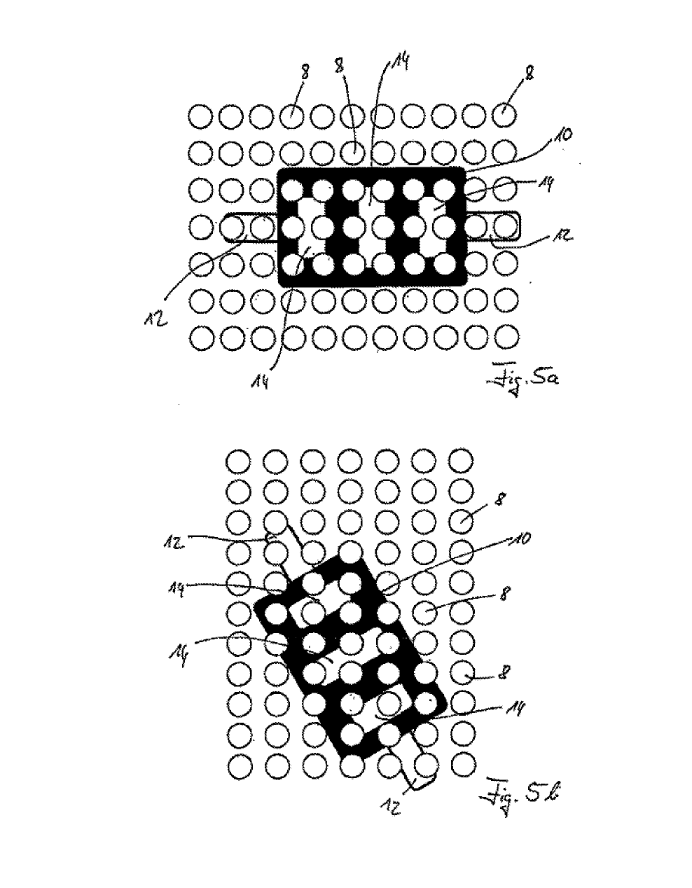

FIG. 4 shows the schematic depiction of a liner and an electrode for a prosthesis system in accordance with a further exemplary embodiment of the present invention,

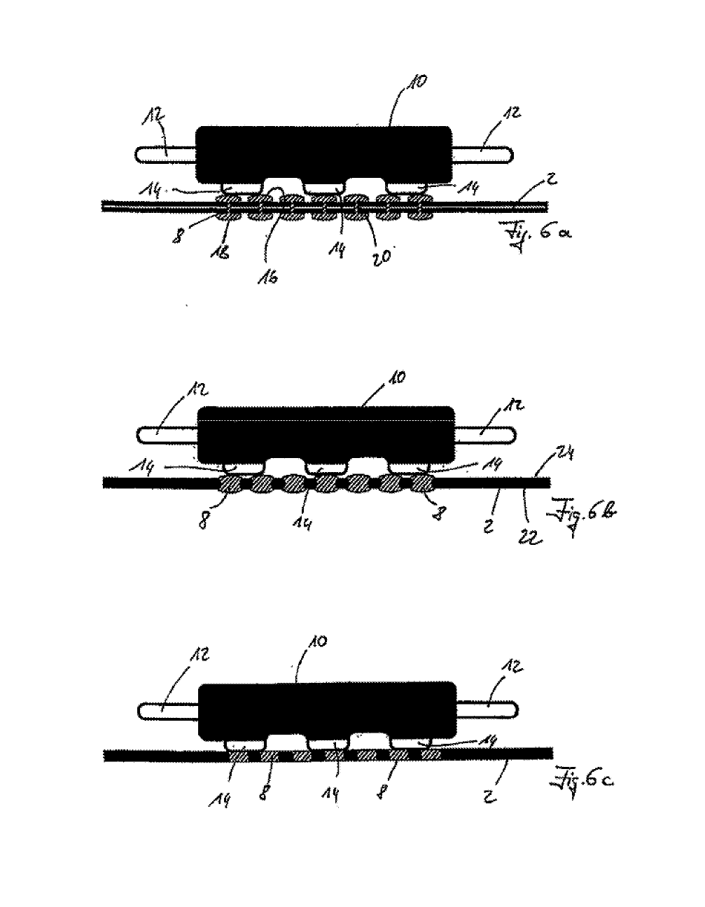

FIGS. 5a, 5b show the arrangement of an electrode relative to a plurality of feedthroughs,

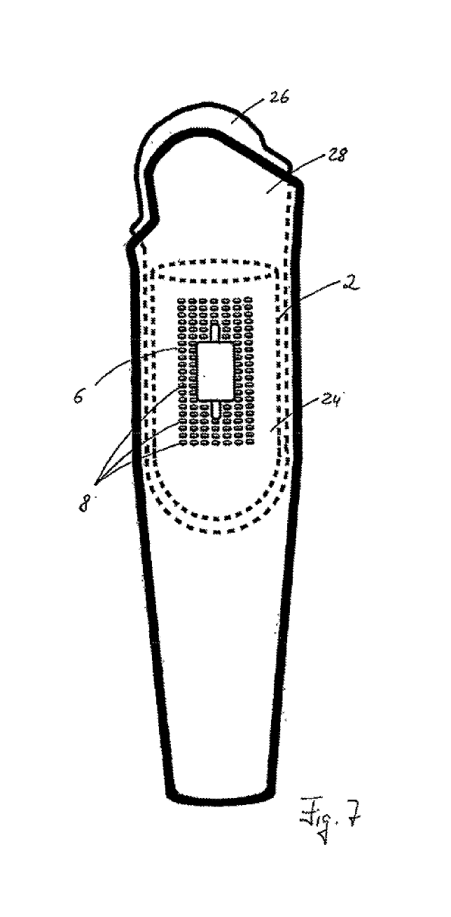

FIGS. 6a, b and c show the schematic depiction of an electrode in contact with a plurality of feedthroughs in a side view and

FIG. 7 shows the schematic depiction of a prosthesis system in accordance with one exemplary embodiment of the present invention.

FIG. 1 shows a liner 2 for a prosthesis system in accordance with one exemplary embodiment of the present invention. The liner 2 consists of an electrically non-conducting liner material, which can, for example, be silicone. On the upper side of the liner 2 in FIG. 1 there is an opening 4 into which the amputation stump is inserted. On the liner 2 there is a region 6 in which a plurality of feedthroughs 8 are arranged.

In the exemplary embodiment shown in FIG. 1, the feedthroughs 8 are arranged in a regular form. As an alternative to this, a free or arbitrary arrangement is also feasible. Here, a distance between two neighboring feedthroughs 8 must in each case be selected to be so large that the feedthroughs 8 do not touch one another since this could lead to short circuit.

FIG. 2 shows the schematic depiction of an electrode 10 for a prosthesis system in accordance with one exemplary embodiment of the present invention. Here, the electrode 10 in FIG. 2 is depicted from below, i.e. from the side resting against the liner. A positioning aid 12, by means of which the electrode 10 can be positioned better and more easily, can be identified in each case on the right-hand and left-hand side. Moreover, provision is made for feed lines (not shown), by means of which the electric signals recorded by the electrode 10 can be transmitted. The electrode 10 shown in FIG. 2 has three contact faces 14, by means of which electric signals can be recorded. In the exemplary embodiment shown in FIG. 2, these are aligned upright next to one another, wherein the central contact face 14 has a larger design than the neighboring contact faces 14. Naturally, other arrangements and numbers of contact faces 14 are also feasible here. The selected number and arrangement depends, in particular, on the functions of the prosthesis system, which should be controlled by the picked up electric signals.

FIG. 3 shows the arrangement of feedthroughs 8, as depicted in FIG. 1 in the region 6 of the liner 2. Shown in the upper region of FIG. 3 is a magnified depiction of a feedthrough 8. The feedthrough 8 comprises an outer contact face 16, which can come into contact with a contact face 14 of an electrode 10. The feedthrough 8 moreover comprises an inner contact face 18, by means of which the feedthrough 8 can come into contact with the skin on the amputation stump of the patient in the applied state of the liner 2. Situated between the outer contact face 16 and the inner contact face 18 there is a feedthrough element 20, which consists of an electrically conducting material and by means of which electric signals can be routed from the inner contact face 18 to the outer contact face 16 and vice versa.

FIG. 4 shows the liner 2 which comprises a plurality of feedthroughs 8 in a region 6. Depicted schematically on these feedthroughs is an electrode 10, as, according to the invention, is arranged on a socket inner face of a prosthesis socket. If the prosthesis socket is pulled over an amputation stump, on which a liner 2 as per FIG. 4 was arranged previously, the electrode 10, which is arranged on the socket inner face of the prosthesis socket, comes into contact with some of the feedthroughs 8, for example in the form shown in FIG. 4.

The liner 2 comprises an inner face 22, by means of which it comes into contact with the amputation stump of the patient, and an outer face 24 facing away from the inner face 22.

FIGS. 5a and 5b schematically show possible arrangements of an electrode 10 relative to a plurality of feedthroughs 8. It is possible to identify that each of the contact faces 14 comes into contact with at least one feedthrough in both arrangements depicted in FIGS. 5a and 5b, and so electric signals which are routed from an inner contact face 18 of a feedthrough 8 to the outer contact face 16 of the feedthrough 8 are recorded by the contact faces 14 of the electrode 10. The electrodes 10 shown here are also embodied with positioning aids 12. Here, as depicted in FIGS. 5a and 5b, it does not matter in which alignment the electrode 10 is arranged relative to the arrangement of the feedthroughs 8. As a result of the distance between two feedthroughs 8 being smaller than the contact face 14, there always is a contact between the contact faces 14 and the outer contact face 16 of the shown feedthroughs 8. The fact that the region 6 in which the feedthroughs 8 are arranged is greater than the extent of the electrode 10 and, in particular, greater than the extent of the contact faces 14 of the electrode 10 ensures that even a displacement, slippage or twist of the liner 2 is harmless for the pickup of the electric signals. Contact is established in each of these cases, and so myoelectric signals can be picked up at the right position, transmitted and processed.

FIGS. 6a, b, and c show the arrangement of an electrode 10 relative to feedthroughs 8 and the liner 2 in a schematic sectional or side view. It is possible to identify that the electrode 10 comprises three contact faces 14, which each come into contact with an outer contact face 16 of a feedthrough 8. FIG. 6a in particular clearly shows that each outer contact face 16 is connected to an inner contact face 18, associated therewith, of the respective feedthrough 8 via the feedthrough element 20. In FIGS. 6a and 6b, the feedthroughs 8 have a raised design relative to the face of the liner 2. This applies both to the inner face 22 and to the outer face 24 of the liner 2. It is only in FIG. 6c that the inner face 22 and the outer face 24 of the liner 2 are formed in a planar and uniform fashion. The liner 2 respectively comes into contact with the skin on the amputation stump of the patient at the underside of the liner 2, on which the inner face 22 is situated. On the opposite outer face 24, the contact to the contact faces 14 of the electrode 10 is established, as depicted in FIGS. 6a to c. If the inner contact face 18 and the outer contact face 16 of the feedthroughs 8 of a liner 2 have a raised design, as shown in FIGS. 6a and 6b, contact between the outer contact face 16 and the contact face 14 of the electrode 10 is simplified. In an embodiment in accordance with FIG. 6c, where the feedthroughs 8 have an integral but not raised design, there are, in particular, no pressure points on the amputation stump of the patient.

FIG. 7 schematically shows a prosthesis system in accordance with an exemplary embodiment of the present invention. Arranged on an amputation stump (not shown) there initially is the liner 2. Situated thereover is a prosthesis socket which comprises an inner socket 26 and an outer socket 28 arranged thereover. Here, the electrode 10 is preferably arranged on the inside of the inner socket 26 or integrated into the inner socket 26. Where these are actually covered by the outer shaft 28 in FIG. 7, they are illustrated by dashed lines. Situated on the outer face 24 of the liner 2 there is a region 6 with a plurality of feedthroughs 8, on which an electrode 10 is arranged schematically. Here, the electrode 10 is arranged on the inner socket 26, and so a precise alignment of the electrode 10 relative to the amputation stump is possible in an easy manner.

Even if the liner 2 in the configuration shown in FIG. 7 is rotated or displaced, there still is contact between the skin of the patient on the amputation stump, at least one feedthrough 8 and the electrode 10 with its contact faces 14, which are not shown in FIG. 7. It follows that the position at which the electric signals are picked up from the amputation stump is merely determined by the contact faces 14 of the electrode. The exact position of the liner 2 is not important in this case, and so a displacement or twist of the liner 2 is harmless for the picking up of myoelectric signals in particular.

### LIST OF REFERENCE SIGNS

2 Liner

4 Opening

6 Region

8 Feedthrough

10 Electrode

12 Positioning aid

14 Contact face

16 Outer contact face

18 Inner contact face

20 Feedthrough element

22 Inner face

24 Outer face

26 Inner socket

28 Outer socket

1. A prosthesis system with
a liner (2) made of an electrically non-conducting liner material, which is provided to be pulled over an amputation stump and which has an inner face (22) provided to rest against the amputation stump and an outer face (24) facing away from the inner face (22),
at least one electrode (10) which has at least one contact face (14), and
a prosthesis socket, which is provided to be arranged on the amputation stump after the liner (2) was pulled thereover such that a socket inner face faces the outer face (24) of the liner (2),
characterized in that
the at least one electrode (10) is arranged on the outer face (24) of the liner (2) or on the socket inner face of the prosthesis socket,
a plurality of feedthroughs (8) which run from the inner face (22) to the outer face (24) and are made of an electrically conducting material in the liner material are arranged in at least one region (6) of the liner (2) and
the at least one contact face (14) of the at least one electrode (10) is provided to come into contact with at least one feedthrough (8) when the prosthesis socket is arranged on the amputation stump (26) after the liner (2) was pulled over the latter.

2. The prosthesis system as claimed in claim 1, characterized in that the at least one region (6) is greater than the at least one contact face (14) of the at least one electrode (10), preferably at least twice the size thereof.

3. The prosthesis system as claimed in claim 1 or 2, characterized in that a plurality of electrodes (10) are arranged on the socket inner face and the liner (2) has a plurality of regions (6) where a plurality of feedthroughs (8) are provided.

4. The prosthesis system as claimed in claim 1, 2 or 3, characterized in that the plurality of feedthroughs (8) have an integral design with the liner (2).

5. The prosthesis system as claimed in claim 1, 2 or 3, characterized in that the plurality of feedthroughs (8) are rivets introduced into the liner material.

6. The prosthesis system as claimed in one of the preceding claims, characterized in that the at least one electrode (10) is displaceably arranged on the outside (24) of the liner (2) or on the socket inner face.

7. The prosthesis system as claimed in one of the preceding claims, characterized in that the at least one electrode (10) has a plurality of contact faces (14).

8. The prosthesis system as claimed in one of the preceding claims, characterized in that an outer contact face (16) of each feedthrough (8) on the outside (24) of the liner (2) is greater than or equal to 1 mm² for the contact with the at least one contact face (14) of the at least one electrode (10).

9. The prosthesis system as claimed in one of the preceding claims, characterized in that the non-conducting liner material is a hydrophobic material.

10. The prosthesis system as claimed in one of the preceding claims, characterized in that the electrically conducting material of the feedthroughs (8) is a hydrophilic material.

11. A liner (2) for a prosthesis system as claimed in one of the preceding claims, characterized in that an inner contact face (18) of each feedthrough (8) on the inner face (22) of the liner (2) has exactly the same size as the outer contact face (16) of each feedthrough (8) on the outer face (24) of the liner (2).

12. A prosthesis socket (28) for a prosthesis system as claimed in one of claims 1 to 10.
